(19) United States
(12) Patent Application Publication
(10) Pub. No.: US 2004/0172113 A1
(43) Pub. Date: Sep. 2, 2004

(54) Tube unit and a blood pump system

(76) Inventors: Kenji Yamazaki, Tokyo (JP); Toshio Mori, Nagano (JP); Ikuo Wakabayashi, Nagano (JP)

Correspondence Address: HOGAN & HARTSON L.L.P., 500 S. GRAND AVENUE, SUITE 1900, LOS ANGELES, CA, 90071-2611, US

(21) Appl. No.: 09/995,246

(22) Filed: Nov. 26, 2001

(51) Int. Cl. A61N 1/00

(52) U.S. Cl. 607/115

(57) ABSTRACT

A tube unit of the present invention is a tube unit used for connecting internal equipment and external equipment, and includes an inner tube which lets liquid flow between the internal equipment and the external equipment, a cable including an electric wire connected to the internal equipment, and an outer tube which accommodates the inner tube and the cable. For the tube unit of the present invention, the expression “internal equipment” refers to a part of equipment, such as an artificial internal organ that can take the place of an internal organ of a living body or an artificial assist device that can assist the functioning of a living body, that is implanted in a living body, or equipment that is implanted in a living body by medical treatment. The expression “external equipment” can refer to equipment, out of equipment that is used as artificial internal organs, a ventricular assist device, or devices used in medical treatment, that is placed outside of a living body. With the tube unit of the present invention, the inner tube and the cable are enclosed within an outer tube, so that the inner tube and the cable are not directly stretched or bent due to movements of the living body or the application of an external force. This protects the inner tube from deformation and the cable from breakages. When the tube unit of the present invention is used for internal equipment such as artificial internal organs, biocompatible materials only need to be used for the outer tube and not for either of the inner tube and the cable, making the tube unit economical. Also, the tube unit of the present invention has the inner tube and outer tube accommodated in the outer tube, so that only one entry point into the living body is required, thereby minimizing the effect of such equipment on the living body.

### BACKGROUND OF THE INVENTION

1. Field of the Invention

The present invention relates to a tube unit for connecting equipment (hereafter referred to as the “internal equipment”) that is implanted in a living body and equipment (hereafter referred to as the “external equipment”) that is located outside the living body. The present invention also relates to a tub unit that is used to connect a blood pump and a controller that controls the blood pump. The present invention further relates to an artificial internal organ system and a blood pump system that include such a tube unit.

2. Background Art

Internal equipment and external equipment are conventionally connected by tubes and cables that are separately provided between the internal equipment and the external equipment. When this method is used, an excessive bending or pulling at the connecting parts of the tubes or cables due to the application of an external force can cause deformation in the tubes and can cause the cables to break. This creates problems with the connections between the internal equipment and the external equipment, which affect the functioning of the internal equipment. When tubes and cables are used to connect an artificial internal organ, each of such tubes and cables contact the tissue of the living body. As a result, each tube and cable needs to be made using a biocompatible material. Also, when a tube suffers deformation or a cable breaks, there is the further problem of the risk of life-threatening injury.

However, to overcome the problems described above, a tube unit that encloses the tubes and cables in another tube has not yet been developed. Such a tube unit can protect the tubes from deformation and the cables from breakages due to the application of an external force, and so can stop problems occurring for the connections between the internal equipment and the external equipment. Also yet to be developed is a tube unit where only an outer tube enclosing the tubes and cables is made using a biocompatible material, thereby making the tube unit compact and limiting the effect of the connections on a living body.

The present invention was conceived to overcome the problems described above, and has an object of providing a compact tube unit where tubes and cables for connecting internal equipment and external equipment are collectively enclosed in a separate tube so as to avoid deformations of the tubes and breakages in the cables due to the application of an external force, the separate tube being produced using a biocompatible material so as to lessen the effects of the tube unit on a living body and the tube unit stopping the movement of the living body and external forces from affecting the living body or the functioning of the internal equipment.

### SUMMARY OF THE INVENTION

The tube unit of the present invention is used for connecting internal equipment and external equipment, and includes an inner tube which lets liquid flow between the internal equipment and the external equipment, a cable including an electric wire connected to the internal equipment, and an outer tube which accommodates the inner tube and the cable.

For the tube unit of the present invention, the expression “internal equipment” can refer to a part of equipment, such as an artificial internal organ that can take the place of an internal organ of a living body or an artificial assist device that can assist the functioning of an internal organ of a living body, that is implanted in a living body, or equipment that is implanted in a living body by medical treatment. The expression “external equipment” can refer to equipment, out of equipment that is used as artificial internal organs, an artificial assist device, or equipment used in medical treatment, that is placed outside of a living body.

With the tube unit of the present invention, the inner tube and the cable are enclosed within an outer tube, so that the inner tube and the cable are not directly stretched or bent due to movements of the living body or the application of an external force. This protects the inner tube from deformation and the cable from breakages.

For the tube unit of the present invention, it is especially preferable for the outer tube to be made of a biocompatible material. It is also preferable for a biocompatible material to be used for the inner tube.

When the tube unit of the present invention is used with internal equipment such as an artificial internal organ, it is not necessary to use a biocompatible material for at least the cable. Also, with the tube unit of the present invention, only one entry point into the living body is required, thereby lessening the influence on the living body.

With the tube unit of the present invention, the cable that is enclosed in the outer tube can include a power cable for driving the internal equipment or a cable that can transmit a signal for controlling the internal equipment and/or a signal that has been detected by the internal equipment.

With the tube unit of the present invention, the inner tube enclosed in the outer tube can be a tube for supplying medication to an affected part, a tube for supplying the internal equipment with a lubricant and/or coolant that is/are required for proper operation of the internal equipment, a tube for circulating blood, or a tube for circulating air, or the like.

With the tube unit of the present invention, more than one cable and more than one inner tube may be enclosed in the outer tube. Even when a plurality of cables and inner tubes are used, there is still only one entry point into the living body, which lessens the influence of such cables and inner tubes on the living body.

For the tube unit of the present invention, the inner tube can form a closed channel through which a liquid can flow. As a result, the circulation of a coolant for the internal equipment or the circulation of a lubricant can be performed efficiently.

A wire to prevent elongation of the inner tube and the cable may also be enclosed on the inside of the tube unit of the present invention. When this construction is used, elongation of the inner tube and the cable is prevented by the wire, so that deformation of the inner tube and breakages of the cable can be effectively prevented.

The tube unit of the present invention may also include caps that are respectively attached to an outside of an engaging part where one end of the outer tube engages a socket for the internal equipment and to an outside of an engaging part where another end of the outer tube engages a socket for the external equipment. When this construction is used, the parts where the outer tube engages each socket are protected by the caps, so that problems, such as the outer tube being pulled off due to movement of the body or the application of an external force, can be avoided.

The tube unit of the present invention may also include protective tubes that engage the caps, are formed of an elastic material, and are attached to an outside of the outer tube. When this construction is used, the protective tubes protect the outer tube against extreme bending, so that the movement of the body or the application of an external force can be prevented from affecting the inner tubes and the cable inside the outer tube.

The outer tube of the present invention is preferably used for connecting the internal equipment and external equipment in an artificial internal organ system including internal equipment implanted in a living body and external equipment located outside the living body.

The expression “artificial internal organ system” here refers to equipment such as an artificial internal organ or artificial assist device, (e.g., a blood pump provided inside a living body to assist the functioning of the heart), a driving apparatus for driving the artificial internal organ or artificial assist device, a cooling apparatus for suppressing heat generated by the driving apparatus, a control apparatus for controlling the operation of the other apparatuses, a filter apparatus for removing impurities and the like from blood that is circulating, a monitor apparatus for monitoring the operating states of the other apparatuses, a warning apparatus for reporting abnormalities when they occur, and a communication apparatus for informing a doctor or a hospital). By collectively enclosing the tubes and cables for connecting these apparatuses in a single tube unit, a large decrease in the effect on a living body of such tubes and cables can be achieved.

Another tube unit according to the present invention is a tube unit used for connecting a blood pump and a controller for controlling the blood pump, including an inner tube for circulating a liquid between the blood pump and the controller, a cable including an electric wire connected to the blood pump, and an outer tube which accommodates the inner tube and the cable.

With this other tube unit of the present invention, the inner tube and cables are protected against elongation and bending due to movement of the living body or the application of an external force, thereby preventing deformation of the inner tube and breakages of the cable.

For this other tube unit of the present invention, it is especially preferable for the outer tube to be made of a biocompatible material. It is also preferable for a biocompatible material to be used for the inner tube. However, with the other tube unit of the present invention, it is not necessary to use a biocompatible material for at least the cable, making the tube unit economical.

With this other tube unit of the present invention, the inner tube and the cable are enclosed in the outer tube, so that only one entry point into the living body is required. This lessens the influence of tube and the cable on the living body.

With this other tube unit of the present invention, more than one cable and more than one inner tube may be enclosed in the outer tube. Even when a plurality of cables and inner tubes are used, there is still only one entry point into the living body, which lessens the influence of such cables and inner tubes on the living body.

It is preferable for this other tube unit of the present invention to include two inner tubes. When two inner tubes are included, there is a definite potential for circulating a liquid between the blood pump and the controller.

With this other tube unit of the present invention, it is preferable for the liquid circulated by the inner tube to be one of water, a disinfectant, and a physiological saline solution. These liquids can function as a coolant for a motor unit in the blood pump, a lubricant for sliding parts, and as a sealant for forming a seal between the blood pump unit and the motor unit. By circulating such liquid between the blood pump and the controller, even if blood seeps into the motor, such blood is diluted by the circulating liquid, making it possible to effectively prevent the motor from stopping due to the coagulation of blood. By filtering the liquid, blood constituents can be removed, making the prevention of the motor stopping even more effective. Circulating a liquid is also effective in dissipating the heat produced in the blood pump.

With this other tube unit of the present invention, it is preferable for the inner tube to be made of polycarbonate urethane, silicone, or polytetrafluoroethylene. Also, with the tube unit of the present invention, it is also preferable for the inner tube to be one of a double-layer tube that has polyvinylidene fluoride on an inside and thermoplastic polyurethane on an outside and a double-layer tube that has polyvinylidene fluoride on an inside and polycarbonate urethane on an outside. When the inner tube is formed using such highly biocompatible materials, even if the circulating liquid is expelled from the blood pump into the living body or if blood or another bodily fluid becomes mixed with the circulating liquid, the occurrence of a thrombus or coagulation of blood can be effectively avoided.

With this other tube unit of the present invention, the electric wire may be one of an electric wire for driving the blood pump and a electric wire for transmitting a signal for controlling the blood pump and/or a signal detected at the blood pump.

With this other tube unit of the present invention, it is preferable for the outer tube to be made of a biocompatible material, with polycarbonate urethane being especially preferable.

With this other tube unit of the present invention, it is preferable for the surface of the outer tube to be subjected to a flocking process. When the surface has been flocked, it becomes easy for the living body and the tube unit to adhere to one another, which is effective in preventing infections from occurring by stopping bacteria from getting between the living body and the outer tube. This also makes it difficult for the tube unit to be pulled out of the living body. Here, the expression “flocking process” refers to covering the outside of the outer tube with flocking (for example, polyester fabric (cloth)).

With this other tube unit of the present invention, it is preferable for the inside of the outer tube to be filled with silicone gel. By doing so, liquid that is circulating in the inner tube and has passed through the material of the inner tube can be effectively prevented from evaporating and dispersing.

The blood pump system of the present invention includes a blood pump, a controller for controlling the blood pump, and the other tube unit described above.

As a result the blood pump system of the present invention has the effects of the blood pump described above, making it a superior blood pump system.

### BRIEF DESCRIPTION OF THE DRAWINGS

FIG. 1 shows the appearance of a tube unit according to a first embodiment of the present invention.

FIG. 2A is a partial cross-sectional view of the internal equipment-end of a tube unit according to the first embodiment of the present invention and FIG. 2B shows a cross-section taken along the line A-A.

FIG. 3A is a partial cross-sectional view of the external equipment-end of a tube unit according to the first embodiment of the present invention and FIG. 3B shows a cross-section taken along the line B-B.

FIG. 4 shows the appearance of a blood pump system according to a second embodiment of the present invention.

FIG. 5 is a cross-sectional figure showing a tube unit according to a second embodiment of the present invention.

FIG. 6 is a cross-sectional figure showing another tube unit according to a second embodiment of the present invention.

### DESCRIPTION OF THE PREFERRED EMBODIMENTS

First Embodiment

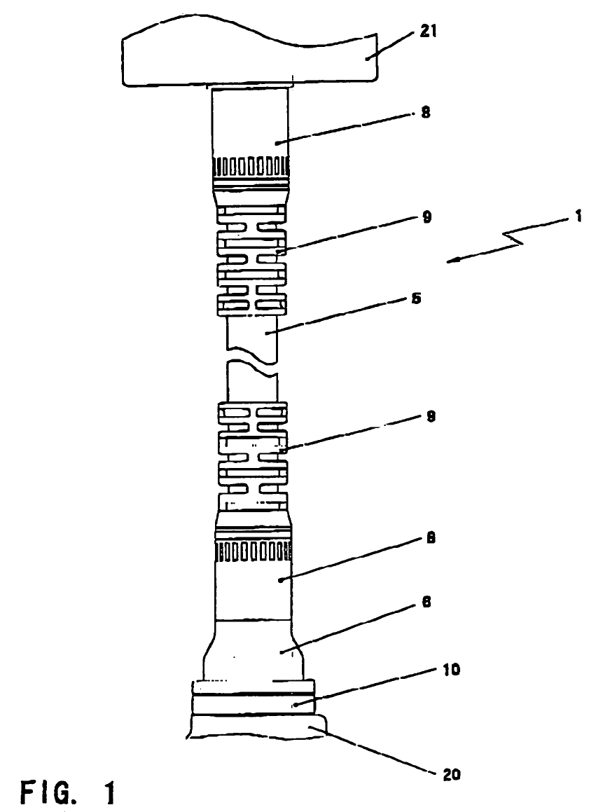

FIG. 1 shows the appearance of a tube unit according to a first embodiment of the present invention. As shown in FIG. 1, a tube unit 1 includes an outer tube 5 that internally encloses a plurality of inner tubes, a power cable and a wire (that are not shown in the drawing), an internal equipment-end socket 6 that holds the outer tube 5, a external equipment-end socket (not shown in the drawings), caps 8 that are attached to an outside of the outer tube 5 at positions where the outer tube 5 engages the respective sockets, protective tubes 9 that engage the caps 8, are attached to the outside of the outer tube 5 and are made of an elastic material, an internal equipment-end connecting member 10 that engages the socket 6 and connects the tube unit 1 to internal equipment 20, and external equipment 21 that connects to an external equipment-end connecting member that engages the socket that is not shown in the drawings.

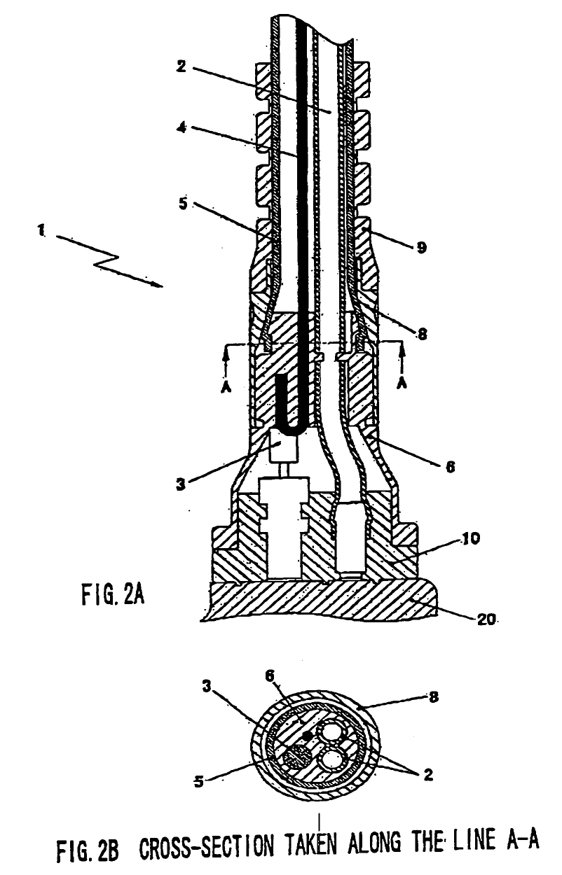

FIG. 2A is a partial cross-sectional view of the internal equipment end of a tube unit according to the first embodiment of the present invention and FIG. 2B shows a cross-section taken along the line A-A. In FIG. 2A and FIG. 2B component 1 is a tube unit, components 2 are a plurality of inner tubes for allowing a liquid to flow between the internal equipment and the external equipment, component 3 is a power cable for supplying electrical power to the internal equipment, component 4 is a wire for preventing elongation of the plurality of inner tubes 2 and the power cable 3, component 5 is an outer tube for collectively enclosing the plurality of inner tubes 2, the power cable 3, and the wire 4, component 6 is an internal equipment-end socket for engaging the end of the wire 4, for holding the plurality of inner tubes 2 and the power cable 3, and engaging the outer tube 5, component 8 is a cap that is attached to an outside of the outer tube 5 at a part where the socket 6 engages the outer tube 5, component 9 is a protective tube that engages the cap 8, is attached to the outside of the outer tube 5, and is made of an elastic material, component 10 is a connecting member that engages the plurality of inner tubes 2 and the power cable 3 and is attached to the socket 6, and component 20 is the internal equipment that is connected to the connecting member 10.

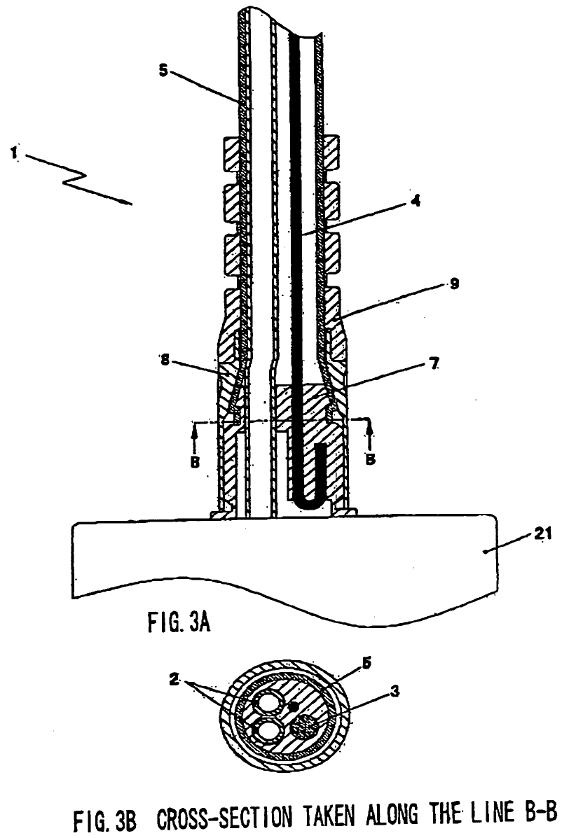

FIG. 3A is a partial cross-sectional view of the external equipment end of a tube unit according to the first embodiment of the present invention and FIG. 3B shows a cross-section taken along the line B-B. In FIG. 3A and FIG. 3B, component 1 is a tube unit, components 2 are a plurality of inner tubes for allowing a liquid to flow between internal equipment and external equipment, component 3 is a power cable for supplying electrical power to the internal equipment, component 4 is a wire for preventing elongation of the plurality of inner tubes 2 and the power cable 3, component 5 is an outer tube for collectively enclosing the plurality of inner tubes 2, the power cable 3, and the wire 4, component 7 is an external equipment-end socket for engaging the end of the wire 4, for holding the plurality of inner tubes 2 and the power cable 3, and engaging the outer tube 5, component 8 is a cap that is attached to an outside of the outer tube 5 at a part where the socket 7 engages the outer tube 5, component 9 is a protective tube that engages the cap 8, is attached to the outside of the outer tube 5, and is made of an elastic material, and component 21 is external equipment that is connected to a connecting member (not shown in the drawings). It should be noted that a connecting member that is attached to the socket 7 and connects to the external equipment 21 is not shown in the drawings.

The plurality of inner tubes 2, which are provided so as to allow liquids and the like to flow between the internal equipment and the external equipment, are formed of a biocompatible resin (such as polycarbonate urethane resin) so that they can pass through internal equipment, such as an artificial internal organ, and so do not affect the living body. Also, since the plurality of inner tubes 2 form a closed channel through which a liquid can flow, they can be used to circulate a gas or liquid, such as oxygen or liquid medication, that is to flow internally between the internal equipment and the external equipment, making the inner tubes 2 highly suitable for use during medical treatment.

The power cable for supplying electrical power to the internal equipment is enclosed in a tube formed of polyvinyl chloride (PVC) resin so that the power cable is protected from the outside. The plurality of inner tubes 2 and the power cable are collectively enclosed within the outer tube, so that only the outer tube that contacts the living body needs to be made using a biocompatible resin and only one entry point into the living body is required. A wire is also enclosed within the outer tube together with the plurality of inner tubes 2 and the power cable 3, with this wire suppressing the bending of the plurality of inner tubes 2 and the power cable 3 so that deformation of the inner tubes and the power cable can be prevented.

One end of the outer tube engages an internal equipment-end socket, while the other end engages an external equipment-end socket. The plurality of inner tubes and the power cable inside the outer tube are held within through-holes that are provided in each of the sockets to allow the inner tubes and the power cable to pass through the sockets, while the wire engages engaging holes provided in the sockets. By having the wire engage the sockets, deformation of the plurality of inner tubes and the power cable is prevented in a lengthwise direction, thereby preventing elongation of the plurality of inner tubes and breakages of the power cable.

Caps are respectively attached to the outside of the part where the outer tube engages the internal equipment-end socket and the outside of the part where the outer tube engages the external equipment-end socket. With this construction, the caps protect the outsides of the parts where the outer tube engages the sockets at the internal equipment and external equipment ends against the effects of the movements of the body and external forces, so that the outer tube can be kept from coming off the sockets. The internal equipment-end socket and the external equipment-end socket are formed of a biocompatible metal, such as titanium, so that connections between the outer tube and the sockets at the internal equipment-end and the external equipment-end can be strongly protected without affecting the living body.

Protective tubes, which are made of an elastic material, have grooves to prevent bending, and engage the caps, are attached to an outside of the outer tube near the parts where the outer tube engages the sockets at the internal equipment-end and the external equipment end. These protective tubes are attached so that the elasticity of the protective tubes prevents excessive bending of the outer tube due to the outer tube being bent near the connections with the sockets. This makes it possible to avoid damage to the plurality of inner tubes and the power cable provided inside the outer tube due to bending caused by movement of the living body or the application of an external force.

The ends of the plurality of inner tubes and the power cable that are held by the internal equipment-end socket and the external equipment-end socket respectively engage an internal equipment-end connecting member and an external equipment-end connecting member. In this way, internal equipment, such as an artificial heart or other artificial internal organ, connected to the internal equipment-end connecting member and external equipment, such as a sub-controller, connected to the external equipment-end connecting member are connected, so that liquids and the like can flow between the internal equipment and the external equipment and so that electrical power can be supplied to the internal equipment. The internal equipment-end socket and external equipment-end socket respectively engage the internal equipment-end connecting member and the external equipment-end connecting member, with the liquid-circulating tubes and power cable that pass through the sockets being covered to protect the tubes and power cable from deformations caused by external factors. As described above, the plurality of inner tubes and the power cable are collectively enclosed in the outer tube with a wire that prevents deformation of the plurality of inner tubes and the power cable in a lengthwise direction, so that a compact tube unit in which all of the required components are enclosed in a small space can be realized.

By enclosing the components (such as tubes and cables for connecting components like (a) an artificial organ or an organ assist device, examples of such being an artificial heart or a ventricular assist device that is provided in a living body to assist the functioning of the heart, (b) a driving apparatus for driving the artificial organ or organ assist device, (c) a cooling apparatus for suppressing heat generation by the driving apparatus, (d) a control apparatus for controlling the operation of these apparatuses, (e) a filter apparatus for removing impurities and the like from blood which is circulating, (f) a monitor apparatus for monitoring the operating states of these apparatuses, (g) a warning apparatus for reporting abnormalities when they occur and (h) a communication means for informing a doctor or a hospital), in a single tube unit, a significant effect is achieved in that the inconvenience caused for the living body is greatly reduced.

As described above, the tube unit of the first embodiment has inner tubes and a power cable collectively enclosed in an outer tube, so that there is no need to use biocompatible materials for the inner tubes and the power cable, and only the outer tube that contacts the living body needs to be made using a biocompatible material. Since the inner tubes form a closed channel through which a liquid can flow, a liquid that flows within the living body can be circulated between the internal equipment and the external equipment, making the tube unit ideal for medical treatment.

Also, a wire that engages the sockets is enclosed in the outer tube along with the inner tubes and the power cable, with the wire preventing elongation of the inner tubes and the power cable thereby preventing deformation in the inner tubes and breakages in the power cable due to elongation. Caps are provided over the parts where the outer tube engages the sockets at the internal equipment-end and the external equipment-end so that the engaging parts can be protected and the outer tube can be kept from coming off the sockets due to the effects on the engaging parts of movements of the body in which the internal equipment is implanted or an external force.

In addition, the internal equipment-end socket and external equipment-end socket that engage the outer tube are formed of a biocompatible metal, such as titanium, so that the parts where the outer tube engages the internal equipment-end socket and the external equipment-end socket can be strengthened without affecting the living body. Furthermore, protective tubes, which have grooves to prevent bending and engage the caps, are attached to an outside of the outer tube near the parts where the outer tube engages the sockets at the internal equipment-end and the external equipment-end. These protective tubes are attached so that the elasticity of the protective tubes prevents excessive bending of the outer tube due to the outer tube being bent near the parts where the outer tube engages the sockets. This means that the damage to the plurality of inner tubes and the power cable provided inside the outer tube due to bending caused by movement of the living body or the application of external forces can be avoided, thereby reducing the effects of such bending on the living body.

Second Embodiment

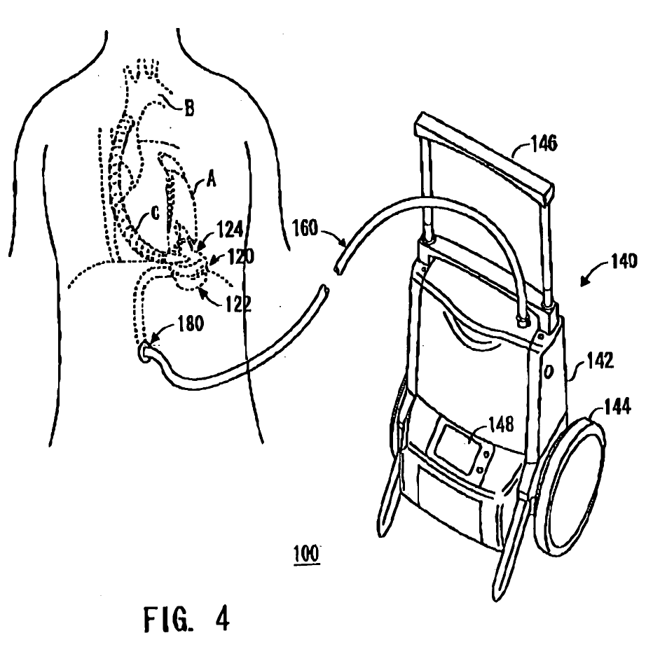

FIG. 4 shows the appearance of a blood pump system 100 according to a second embodiment of the present invention. As shown in FIG. 4, the blood pump system 100 of this second embodiment includes a blood pump 120, a controller 140 for controlling the blood pump 120, and a tube unit 160 that is used to connect the blood pump 120 and the controller 140.

The blood pump 120 and the controller 140 in the blood pump system 100 are fundamentally the same as those shown in FIGS. 1, 2, 3, 4, 6 and 7 of U.S. Pat. No. 6,123,726 and described in the relevant parts of the specification. Accordingly, the disclosure of U.S. Pat. No. 6,123,726 is incorporated by reference into the present specification.

As described in the cited US patent, the blood pump 120 includes a pump base section 122, which has a cylindrical motor, and a pump section 124, which is connected to the pump base section 122. The pump section 124 includes pump vanes that are driven via a rotational shaft of the motor and a casing that is connected to the pump base section 122 so as to cover the pump vanes. Blood in the left ventricle A flows into the casing from an intake provided at the end of the casing, the blood is energized in the casing by the pump vanes, and the blood is then expelled into the aorta B via an outtake provided in the side of the casing and an artificial vessel C.

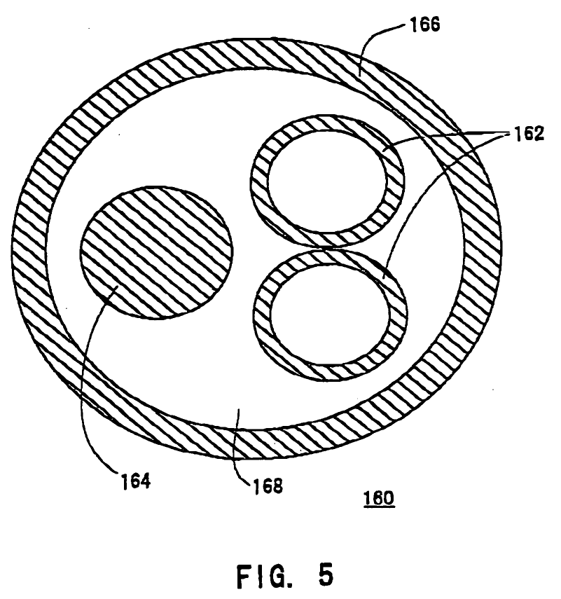

An end-contact type blood seal (hereafter also referred to as a “mechanical seal”) is provided between the pump base section 122 and the pump vanes, so as to stop blood constituents seeping in and coagulating in the bearings of the rotational shaft of the motor. The pump base section 122 is also provided with an intake and an outtake for a circulating fluid, with the intake and the outtake for the circulating fluid being connected to the controller 140 via inner tubes 162 (see FIG. 5) enclosed within the tube unit 160.

The controller 140 is a circulating liquid pump that circulates the circulating liquid to the periphery of the mechanical seal. As a result, lubrication, cooling, and dispersion occur at the sliding surfaces of the mechanical seal. In addition, a filter provided in the controller 140 removes fine particles of blood constituents that enter the circulating fluid so as to constantly keep the sliding surfaces of the mechanical seal and the bearings clean.

A centrifugal pump, an axial-flow pump, a mixed-flow pump, or the like can be favorably used as the blood pump 120. Also, the blood seal is not limited to a mechanical seal, so that another type of contact seal, such as an oil seal, may be used.

The controller 140 is also as described in the cited US patent, and so has a system driving section, composed of a circulating pump for supplying circulating liquid to the periphery of the mechanical seal in the blood pump 120, a pump control unit for electrically controlling the driving of the blood pump 120 via a cable 164 enclosed in the tube unit 160, a display unit 130 for displaying data and the operating states of the various components, a communication unit for exchanging information with external devices, a power supplying unit for supplying these components with electrical power, and a control unit for controlling these components. The system driving section is enclosed in a compact case 142 and is placed on a mobile controller 140 that includes wheels 126 and a handle 128.

Also, while the controller 140 in the present embodiment is in the form of a cart that is pushed by hand, the controller may be alternatively produced in the form of a wheelchair or a bag, so that a form that is suited to the condition and living arrangements of the patient may be used. An electric mobile controller 140 may also be used by attaching a motor to the wheels 144.

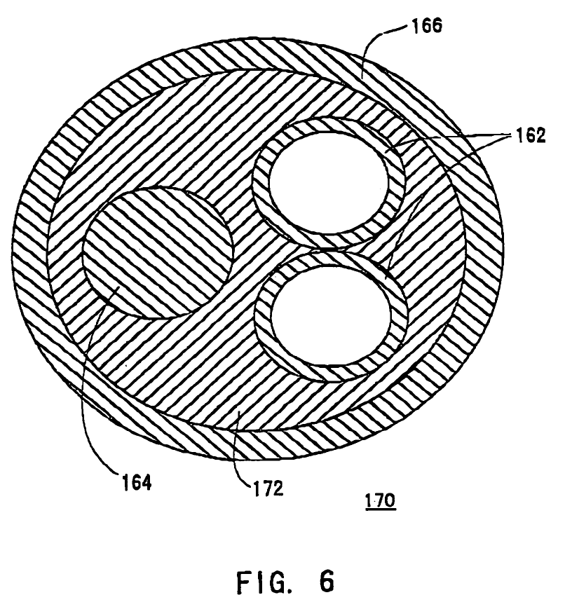

FIG. 5 is a cross-sectional drawing showing the tube unit 160 according to this second embodiment, while FIG. 6 is a cross-sectional drawing showing a different tube unit 170 according to this second embodiment.

As shown in FIGS. 4, 5, and 6, the tube unit 160 according to the second embodiment is a tube unit used to connect the blood pump 120 and a controller 140 that controls the blood pump 120. The tube unit 160 includes inner tubes 162 for circulating a liquid between the blood pump 120 and the controller 140, a cable 164 that internally includes electrical wires for connecting the blood pump 120, and an outer tube 166 for enclosing the inner tubes 162 and the cable 164.

As a result, with the tube unit 160 according to the second embodiment, the inner tubes 162, the cable 164, etc., are unaffected by stretching and bending caused by movements of the living body or the application of an external force, so that the inner tubes 162 are protected against deformation and the cable 164 is protected against breakages.

With the tube unit 160 according to the second embodiment, only one entry point (shown as a support 180 in FIG. 4) into the living body is required, which can minimize the influence on the living body.

With the tube unit 160 according to the second embodiment, biocompatible materials are used for the inner tubes 162 and the outer tube 166.

The tube unit 160 according to the second embodiment has two inner tubes 162, with these two inner tubes 162 being used to circulate pure water between the blood pump 120 and the controller 140. The pure water acts as a coolant for the motor unit in the blood pump, a lubricant for the sliding parts of the blood seal, and a sealant that provides a seal between the motor unit and the blood pump unit. Also, by having pure water circulated between the blood pump and the controller, the motor can be effectively prevented from coming to a stop since any blood that enters the motor is diluted by the pure water, which stops the blood from coagulating. The pure water is also filtered, so that any blood constituents that enter the pure water can be removed, thereby making the system even more effective at preventing blood from stopping the rotation of the motor. Also, by having the pure water circulated, heat inside the blood pump can be effectively dissipated.

In the tube unit 160 according to the second embodiment, double-layer tubes that have polyvinylidene fluoride on the inside and thermoplastic polyurethane on the outside are used as the inner tubes 162. Since polyvinylidene fluoride is a highly biocompatible material, even if pure water is expelled from the blood pump into the living body or if blood or another bodily fluid becomes mixed with the pure water, the occurrence of a thrombus or coagulation of blood can be effectively avoided.

The tube unit 160 according to the second embodiment includes one cable 164, though this cable 164 includes three-phase/three-wire electrical wiring for powering the rotation of the motor and controlling the speed of rotation.

The tube unit 160 according to the second embodiment uses a tube composed of polycarbonate urethane, a material with favorable biocompatibility, as the outer tube 166.

In the tube unit 160 according to the second embodiment, the surface of the outer tube 166 is subjected to a flocking process using a polyester fabric. As a result, it is easy for the living body to adhere to the tube unit, which is effective in preventing infections from occurring by stopping bacteria from getting between the living body and the outer tube. This also makes it difficult for the tube unit 160 to be pulled out of the living body.

In the tube unit 160 according to the second embodiment, the inside of the outer tube 166, with the exception of the inner tubes 162 and the cable 164, is empty. However, as shown in FIG. 6, the inside of the outer tube 166 may be filled with silicone gel. By doing so, pure water that is circulating in the inner tubes 162 and has passed though the material of the inner tubes 162 can be effectively prevented from evaporating and dispersing.

The blood pump system 100 according to the second embodiment is equipped with a blood pump 120, a controller 140 for controlling the blood pump 120, and a tube unit 160 for circulating a liquid between the blood pump 120 and the controller 140. As a result, the blood pump system 100 has the effects of the tube unit 160 that are described above.

It should be noted that the blood pump system 100 according to the second embodiment does not include the connecting terminals that are shown in FIGS. 1 and 2 of U.S. Pat. No. 6,123,726. As a result, the blood pump system according to the second embodiment is a highly reliable system that does not suffer from poor connections caused by the connecting terminals.

What is claimed is:

1. A tube unit used for connecting internal equipment and external equipment, comprising:
an inner tube which lets liquid flow between the internal equipment and the external equipment;
a cable including an electric wire connected to the internal equipment; and
an outer tube which accommodates the inner tube and the cable.

2. A tube unit according to claim 1,
wherein the internal equipment is one of (a) a part of one of artificial internal organs and a ventricular assist device that is implanted in a living body and (b) equipment that is implanted in a living body by medical treatment.

3. A tube unit according to claim 1,
wherein the external equipment is equipment, out of equipment that is used as one of artificial internal organs, a ventricular assist device, and equipment used in medical treatment, that is placed outside of a living body.

4. A tube unit according to claim 1,
wherein the cable is one of a power cable for driving the internal equipment and a cable for transmitting a signal for controlling the internal equipment and/or a signal detected by the internal equipment.

5. A tube unit according to claim 1,
wherein the inner tube is one of a tube for supplying medication to an affected part and a tube for supplying the internal equipment with a lubricant and/or a coolant, which is/are required for proper operation of the internal equipment.

6. A tube unit according to claim 1,
wherein a channel through which a liquid can flow is formed by the inner tube.

7. A tube unit according to claim 1,
wherein a wire for preventing elongation of the inner tube and the cable is accommodated in the outer tube.

8. A tube unit according to claim 1,
further comprising caps that are respectively attached to an outside of an engaging part where one end of the outer tube engages a socket for the internal equipment and to an outside of an engaging part where another end of the outer tube engages a socket for the external equipment.

9. A tube unit according to claim 1,
further comprising protective tubes that engage the caps, are formed of an elastic material, and are attached to an outside of the outer tube.

10. An artificial internal organ system, including a tube unit according to any of claims 1 to 9.

11. A tube unit used for connecting a blood pump and a controller for controlling the blood pump, comprising:
an inner tube for circulating a liquid between the blood pump and the controller;
a cable including an electric wire connected to the blood pump; and
an outer tube which accommodates the inner tube and the cable.

12. A tube unit according to claim 11,
wherein the tube unit comprises two inner tubes.

13. A tube unit according to claim 11,
wherein the liquid circulated by the inner tube is one of water, a disinfectant, and a physiological saline solution.

14. A tube unit according to claim 11,
wherein the inner tube is made of one of polycarbonate urethane, silicone, and polytetrafluoroethylene.

15. A tube unit according to claim 11,
wherein the inner tube is one of a double-layer tube that has polyvinylidene fluoride on an inside and thermoplastic polyurethane on an outside and a double-layer tube that has polyvinylidene fluoride on an inside and polycarbonate urethane on an outside.

16. A tube unit according to claim 11,
wherein the electric wire is one of a electric wire for driving the blood pump and a electric wire for transmitting a signal for controlling the blood pump and/or a signal detected at a position of the blood pump.

17. A tube unit according to claim 11,
wherein the outer tube is made of a biocompatible material.

18. A tube unit according to claim 11,
wherein a surface of the outer tube is subjected to a flocking process.

19. A tube unit according to claim 11,
wherein an inside of the outer tube is filled with silicone gel.

20. A blood pump system, comprising:
a blood pump;
a controller for controlling the blood pump; and
a tube unit according to any of claims 11 to 19.
